SEQUENCE LISTING (10 sequences)

SEQ ID NO: 1 - DNA, 2044 bp, Arabidopsis thaliana
ctttcacatctccctctctcttacaactctctatccgtagtttcagtttcgtcctagaatttcttttcttgtctgttccagatgaaattgtgagttctccgagttatatgcatccgttagaatcaaagcggtaatcaaaagcagaatttagaggattgaaaaagatggttttggggtgtttccctttgaaaagcaagaagaaacgtggctctgtttctatgaagcggttggatcttgaagaaagcaagccaactgctttacctgagccaccaaagattccaagtcgtaatttacaatcaggtcctccgagtttcagaaatcgtgtgaagccaattcaatcaaacaacggtggaaacggagagatgagtagccgagcaagagtcatgtttgctccgtcaagcatccacggtgcagcggaacgggatttgcttgctggtgtttaccacgacgagcaagatgaacaaccaagagatccacgtacttctactaaagaatctagccctcaaccacttccgttaccgtcaccaagaactggttcttcattgaagaattggggaagctttaagtcgtttaacggaagcagcggtcggttatcatcatccgcagctgtatctggacctttacctttgccacctagcgggtcagttaggagcttttcatatgatgaagtaatggctgcgtgtaacgctttttcttcagaccgatgtgtcatggaaggtctttcatctgttatgtacatggcttcctttggtgatgaggcttcgacctcaggtttaaagaaggttgacgcaactgttgtacgacttcacgtaactactcagagtattagggagttcattaatgaagtcaacacattggcgtcgctgcaacaccagaacctttgtaagctggtaggctatcatgctcgtgacggttctgacacaagaatgttggtgtacgagaggcttgctctgggcagcttggaccgtttactgcatgggagatcagatgggcctcctcttgattggaacactagaatgaagattgcactatgtgcagctcagggtctaaccttcttgcacgaagaaggcccttttcaggcaatgtacaatgaattttcgacggcaaatatccaagtcgataaagatttcagcgccaagctatcaggatacggttgtgcaggccatgcgcctgagacagagacatctaatagttcggcacttgctaatctctctgtcgagactctagagagagggcttttgaccccgaagagcaatgtgtggagctatggaatagttcttcttgagatgttaacgggtcggaaaaatatggacgggtcttacccgaaagaagagaggaacttagtgaaatggagcagagcttttctagcagatgattgcaggctctcgcttataatggatcctcagcttaaaggtcggtttccggcaaaagcggcgaggagcatagcagatatagcacagaaatgtctgcaggtggagccatcagagcgtccaaccatgagaaacatcgtggatcaactcaagatcatacaggacatgaagtactcgtgtaggttcccgttaagagaacccgcaccagtcgcggcaaggaaacatatgggaagatcaagcagtctcaacacgattatttggaccccggcatcagtgccaccaaggtcaagtttttcaccgtcacctccaccacgacgaccgtctgtttcacccacaaggggacggacgctcgtgtttcccccagtgtttccgccgcgagcgtgttcatctttggaggaaatggctcgggaagaggttcgaagatcgtcttcagccagtggtaggagaactagcctcgaagggttttgatttgttcatatctctcttctttcttctttttcctatttgtttttaatttttggcgttgatttgttttttggattccaaatataaaaaattgtttttttctttttcttttgttgtttatgaatccggacgtcgatagagagggtttgtgatgtattgtacataataatcaacatttcttctttcacattaaaaaaaaaaaaaaaaaa

SEQ ID NO: 2 - protein, 557 aa, Arabidopsis thaliana
MVLGCFPLKSKKKRGSVSMKRLDLEESKPTALPEPPKIPSRNLQSGPPSFRNRVKPIQSNNGGNGEMSSRARVMFAPSSIHGAAERDLLAGVYHDEQDEQPRDPRTSTKESSPQPLPLPSPRTGSSLKNWGSFKSFNGSSGRLSSSAAVSGPLPLPPSGSVRSFSYDEVMAACNAFSSDRCVMEGLSSVMYMASFGDEASTSGLKKVDATVVRLHVTTQSIREFINEVNTLASLQHQNLCKLVGYHARDGSDTRMLVYERLALGSLDRLLHGRSDGPPLDWNTRMKIALCAAQGLTFLHEEGPFQAMYNEFSTANIQVDKDFSAKLSGYGCAGHAPETETSNSSALANLSVETLERGLLTPKSNVWSYGIVLLEMLTGRKNMDGSYPKEERNLVKWSRAFLADDCRLSLIMDPQLKGRFPAKAARSIADIAQKCLQVEPSERPTMRNIVDQLKIIQDMKYSCRFPLREPAPVVARKHMGRSSSLNTIIWTPASVPPRSSFSPSPPPRRPSVSPTRGRTLVFPPVFPPRACSSLEEMAREEVRRSSSASGRRTSLEGF

SEQ ID NO: 3 - DNA, 13 bp, Artificial Sequence
  Description of Artificial Sequence Primer
aattcggcacgag

SEQ ID NO: 4 - DNA, 15 bp, Artificial Sequence
  Description of Artificial Sequence Primer
ttttttttttttttt

SEQ ID NO: 5 - DNA, 24 bp, Artificial Sequence
  Description of Artificial Sequence Primer
gaattcagaggattgaaaaagatg

SEQ ID NO: 6 - DNA, 21 bp, Artificial Sequence
  Description of Artificial Sequence Primer
caaatccaaacccttcgaggc

SEQ ID NO: 7 - DNA, 30 bp, Artificial Sequence
  Description of Artificial Sequence Primer
gctcaggatccgattgtcgtttcccgcctt

SEQ ID NO: 8 - DNA, 29 bp, Artificial Sequence
  Description of Artificial Sequence Primer
gatacaatcgatatcagccaattttagac

SEQ ID NO: 9 - protein, 389 aa, Arabidopsis thaliana
MGGCFSNRIKTDIASSTWLSSKFLSRDGSKGSSTASFSYMPRTEGEILQNANLKNFSLSELKSATRNFRPDSVVGEGGFGCVFKGWIDESSLAPSKPGTGIVIAVKRLNQEGFQGHREWLAEINYLGQLDHPNLVKLIGYCLEEEHRLLVYEFMTRGSLENHLFRRGTFYQPLSWNTRVRMALGAARGLAFLHNAQPQVIYRDFKASNILLDSNYNAKLSDFGLARDGPMGDNSHVSTRVMGTQGYAAPEYLATGHLSVKSDVYSFGVVLLELLSGRRAIDKNQPVVEHNLVDWARPYLTNKRRLLRVMDPRLQGQYSLTRALKIAVLALDCISIDAKSRPTMNEIVKTMEELHIQKEASKEQQNPQISIDNIINKSPQAVNYPRPSIM

SEQ ID NO: 10 - protein, 425 aa, Arabidopsis thaliana
MAVFKKKKTSLTSLFLGCYKAKNASKYEGGEKAVMKIRTCPAFKRLSLSDISDPSSPMSVMDDLSHSFTSQKLRLFTLSELRVITHNFSRSNMLGEGGFGPVYKGFIDDKVKPGIEAQPVAVKALDLHGHQGIENGWRRYYFSGQLSNKHLVKLIGFCCEEEQRVLVYEYMPRGSLENQLFRRNSLAMAYVGIRMKIALGAAKGLAFLHEAEKPVIYRDFKTSNILLDSDYNAKLSDFGLAKDGPEGEHTHVTTRVMGTQGYRAPEYIMTGHLTTMNDVYSFGVVLLELITGKRSMDNTRTRREQSLVEWARPMLRDQRKLERIIDPRLANQHKTEAAQVAASLAYKCLSQHPKYRPTMCEVVKVLESIQEVDIRKHDGNNNKEGKKFVDINKFRHHRKGKRRVNIAYSDSLVYKESKAKQNDGI

(19) United States
(12) Patent Application Publication
(10) Pub. No.: US 2005/0054831 A1
(43) Pub. Date: Mar. 10, 2005

(54) Osmotic stress-inducible protein functioning as a negative regulator in osmotic stress signaling pathway of plants

(76) Inventors: In-Hwan Hwang, Pohang (KR); Jeong-Hwa Lim, Jinjoo (KR); Kyung-Tae Pih, Pohang (KR)

Correspondence Address: REED INTELLECTUAL PROPERTY LAW GROUP, 800 MENLO AVENUE, SUITE 210, MENLO PARK, CA, 94025, US

(21) Appl. No.: 10/239,919

(22) PCT Filed: Feb. 1, 2002
(86) PCT No.: PCT/KR02/00152

(30) Foreign Application Priority Data
Feb. 2, 2001 (KR) 2001/5097

(51) Int. Cl. C07H 21/04; A01H 1/00; C12N 15/82; C07K 14/415

(52) U.S. Cl. 530/370; 435/468; 536/23.6

(57) ABSTRACT

The present invention relates to an AtSIK protein functioning as a negative regulator to the osmotic stress-inducible gene and more particularly to a gene encoding said protein. Said AtSIK protein isolated from Arabidopsis thaliana acts as a negative regulator to the osmotic stress-inducible gene. Therefore, a plant having enhanced resistance to osmotic stress can be produced by inhibiting expression of AtSIK and consequently, the productivity of the plant can be increased.

### TECHNICAL FIELD

The present invention relates to a protein AtSIK functioning as a negative regulator of osmotic stress-inducible genes, a gene encoding the protein and a method for enhancing resistance to osmotic stress in plants by repressing the gene.

### BACKGROUND ART

Osmotic stress is caused by various external conditions such as dehydration, high salinity and cold, and is one of the most severe stresses among environmental stresses which inhibit plant growth. Recent studies have focused on molecular biological approaches to study plant mechanisms responding to such an osmotic stress (Bohnert et al., Plant Cell, 7: 1099-1111 1995; Shinozaki and Yamaguchi-Shinozaki, Curr. Opin. Plant Biol., 3: 17-223, 2000). Actually, many genes that are responsive to the osmotic stresses have been isolated, and their characteristics have been studied (Skriver and Mundy, Plant Cell 2: 503-512, 1990; Pih et al., Mol Cells, 9: 84-90, 1998; Anderberg and Walker-Simmons, Proc. Natl. Acad. Sci. USA, 89: 10183-10187, 1993; Mizoguchi et al., Proc. Natl. Acad Sci. USA, 93: 765-769, 1996). From those studies, it was cleared that there are multiple signal transduction pathways that lead to induction of osmotic stress-responsive genes (Jonak et al., Proc. Natl. Acad. Sci. USA, 93: 11274-11279, 1996; Ishitani, Plant Cell, 9: 1935-1949, 1997), and these pathways include ABA (abscisic acid)-dependent or ABA-independent pathway (La Rosa et al., Plant Physiol., 85: 174-181, 1987; Savoure et al., Mol. Gen. Genet., 254: 104-109, 1997). In addition, it was discovered that some signaling pathways are common to all osmotic stress conditions, while other signaling pathways are specific to certain osmotic stress conditions such as low temperature, high salt and dehydration (Jang et al., Plant Mol. Biol., 37: 839-847, 1998; Liu et al., Science, 280: 1943-1945, 1998; Pardo et al., Proc. Natl. Acad. Sci. USA, 95: 9681-9686, 1998; Liu et al., Proc. Natl. Acad. Sci. USA, 97: 3730-3734, 2000).

Among a variety of osmotic stress-inducible genes, many genes encode protein kinases (Anderberg and Walker-Simmons, Proc. Natl. Acad. Sci. USA, 89: 10183-10187, 1993; Urao et al., Plant Cell, 11: 1743-1754, 1994; Hwang and Goodman, Plant J. 8: 37-43, 1995; Mizoguchi et al., Proc. Natl. Acad. Sci. USA, 93: 765-769, 1996, Munnik et al., Plant J. 20: 381-388, 1999), and they are shown to play important roles in osmotic stress signal transduction pathways.

Therefore, studies at molecular biological levels on those genes require an understanding of the mechanisms of controlling osmotic stress signaling pathways. Heretofore, only a limited number of kinases involved in osmotic stress signaling pathways have been found. MAP kinase cascade is an important signaling pathway involved in responses to osmotic stress in animal and yeast cells (Blumer et al., Proc. Natl. Acad. Sci. USA, 91: 4925-4929, 1994; Bode et al., J. Biol. Chem., 274: 30222-30227, 1999; Maeda et al., Nature, 369: 242-245, 1995; Posas et al., Cell, 86: 865-875, 1996). Some kinase homologues involved in such pathways were isolated from plants such as Arabidopsis thaliana (Mizoguchi et al., Proc. Natl. Acad. Sci. USA, 93: 765-769, 1996; Covic et al., Biochem. Biophys. Acta., 1451: 242-254, 1999), Pisum sativum (Popping et al., Plant Mol. Biol., 31: 355-363, 1996) and alfalfa (Jonak et al., Proc. Natl. Acad. Sci. USA, 93: 11274-11279, 1996).

Those MAP kinase homologues are rapidly activated by osmotic stress conditions such as low temperature, drought and high concentration of salt, and are additionally activated by exogenously applied ABA (absicic acid) when assayed in an in vitro experiment (Foster and Chua, Plant J., 17: 363-372, 1999; Droillard et al., FEBS Lett., 474: 217-222, 2000; Mikolajczyk et al., Plant Cell, 12: 165-178, 2000).

Protein phosphatase 2C type isolated from alfalfa is known as a negative regulator of MAP kinase pathway (Meskiene et al., Proc. Natl. Acad Sci. USA, 95: 1938-1943, 1998), and ATHK1 protein having a structural similarity to the yeast osmo-sensing histidine kinase Sln1p is isolated from Arabidopsis (Urao et al., Plant Cell, 11: 1743-1754, 1999). It was discovered that these proteins serve to initiate signals in response to osmotic stress in plants, similarly to yeast cells.

SOS2 is a gene encoding a kinase which is required to exhibit salt tolerance (Liu et al., Proc. Natl. Acad. Sci. USA, 97: 3730-3734 2000). AtGSK1 isolated from Arabidopsis shows a high degree of similarity to GSK3/shaggy kinase isolated from animal, and the protein is involved in response to salt stress (Piao et al., Plant Physiol., 119: 1527-1534 1999).

However, there are still no clearly identified genes involved in osmotic stress-induced signaling pathways and signaling mechanisms thereof.

### DISCLOSURE OF THE INVENTION

Accordingly, the present inventors have conducted studies to find a novel gene involved in resistance of plants to environmental stress, particularly, osmotic stress. As a result, they found a novel protein, AtSIK, which is isolated from Arabidopsis thaliana, containing a kinase domain and having a function of repressing stress-inducible genes, thereby illuminating a method of enhancing resistance of the plant to osmotic stress by inactivation of AtSIK gene.

Thus, it is an object of the invention to provide a novel protein functioning as a negative regulator of genes involved in resistance to diverse osmotic stresses.

It is another object of the invention to provide a gene encoding the protein functioning as a negative regulator of genes involved in resistance to osmotic stress.

It is yet another object of the invention to provide a method for enhancing resistance of a plant to osmotic stress by inactivating the gene.

In accordance with one aspect of the present invention, the above and other objects can be accomplished by the provision of a protein, AtSIK comprising the amino acid sequence of SEQ ID NO: 2, which is a negative regulator of genes involved in resistance to osmotic stress.

In accordance with another aspect of the present invention, there is provided an AtSIK gene represented by SEQ ID NO: 1 encoding AtSIK, the negative regulator of genes involved in resistance to osmotic stress.

Where a mutation occurs in the AtSIK gene, a plant exhibits resistance to osmotic stress. In contrast, a wild type plant without a mutation in the AtSIK gene exhibits sensitivity to osmotic stress, whereby anthocyanin is accumulated in leaves and leaf edges has been broken, and small and etiolated leaves are seen.

In accordance with yet another aspect of the present invention, there is provided a method for enhancing resistance of a plant to osmotic stress, comprising the step of constructing a transformed plant whose AtSIK gene is inactivated, thereby increasing productivity of the plant.

The term “osmotic stress-inducible gene” or “osmotic stress-responsive gene” as used herein refers to a gene encoding a protein induced by osmotic stress, which is caused by exposure of the plant to high concentration of salt, low temperature, dehydration, or exogenous ABA treatment; or a gene encoding a protein involved in exhibiting tolerance or resistance to osmotic stress.

The term “AtSIK-complement” refers to a recombinant vector comprising an active AtSIK gene, which allows an AtSIK-inactivated mutant to express the AtSIK protein. The term “AtSIK-complemented mutant” refers to a mutant plant transformed with the AtSIK-complement.

Additionally, a description herein referring to a gene encoding AtSIK, a protein, is in italics, that is, “AtSIK gene” or “AtSIK”, while the protein encoded by the gene is represented as “AtSIK protein” or “AtSIK”.

### BRIEF DESCRIPTION OF THE DRAWINGS

The above and other objects, features and other advantages of the present invention will be more clearly understood from the following detailed description taken in conjunction with the accompanying drawings.

FIG. 1 shows a comparison of an amino acid sequence of AtSIK with those of other protein kinases.

FIG. 2 shows a Northern blot analysis of an expression pattern of an AtSIK gene in diverse plant tissues such as flower, leaf, root and silique of Arabidopsis thaliana.

FIG. 3 shows a Northern blot analyses of expression patterns of an AtSIK gene induced by diverse osmotic stresses (the numerals represent treatment time of osmotic stress).

FIG. 4 shows results of PCR screening and Southern blot analysis for selecting and confirming a mutant harboring a T-DNA insert within an AtSIK gene and a location of the T-DNA insert in AtSIK:

A) shows primary, secondary and tertiary PCR products on agarose gels; and

B) shows a location of T-DNA within AtSIK in a T-DNA insertion mutant.

FIG. 5 shows the phenotype of a T-DNA insertion mutant:

A) shows a Southern blot analysis of genomic DNA, which was digested with BamHI, EcoRI, HindIII, and XhoI, of a wild type (WT) and a T-DNA insertion mutant (CD380-2); and

B) shows a Northern blot analysis of expression patterns of an AtSIK gene after application with dehydration, low temperature or high concentration of salt in a wild type (WT) and a T-DNA insertion mutant (CD380-2).

FIG. 6 shows phenotypic changes in wild type (WT) and T-DNA insertion mutant (CD380-2) plants after application with dehydration, low temperature or high concentration of salt:

A) refers to dehydration;

B) refers to 150 mM NaCl treatment;

C) refers to a low temperature of 4° C; and

D) shows a comparison of leaf sizes upon osmotic stress treatment.

FIG. 7 shows cross-sections of leaf tissues under an electron microscope, after wild type (WT) and T-DNA insertion mutant (CD380-2) plants were exposed to low temperature, and their leaf tissues were fine-cut:

A) refers to an untreated wild type;

B) refers to an untreated T-DNA insertion mutant;

C) refers to a wild type upon treatment at 4° C.; and

D) refers to a T-DNA insertion mutant upon treatment at 4° C.

FIG. 8 shows Northern blot analysis of expression patterns of other osmotic stress-inducible genes, COR15a, COR47, AtSIZ, and RD29A, in wild type (WT), T-DNA insertion mutant (CD380-2) and T-DNA-complemented mutant plants (CD380-2:AtSIK 2- 1):

A) shows gene expression induced by treatment at 4° C. in wild type (WT) and T-DNA insertion mutant (CD380-2); and

B) shows gene expression induced by treatment with 150 mM NaCl in wild type (WT), T-DNA insertion mutant (CD380-2) and T-DNA-complemented mutant plants (CD380-2:AtSIK 2-1).

FIG. 9 shows Northern blot analysis of expression patterns of AtSIK in wild type (WT), T-DNA insertion mutant (CD380-2) and AtSIK-overexpressing mutant plants (CD380-2:AtSIK 2-1) (A), and shows NaCl sensitivity in wild type (WT), T-DNA insertion mutant (CD380-2) and AtSIK-overexpressing mutant plants (CD380-2:AtSIK2-1), upon treatment with 0, 0.1, 0.15 and 0.2 mM NaCl, respectively (B).

### BEST MODE FOR CARRYING OUT THE INVENTION

The inventors intended to isolate a novel gene induced by osmotic stress. Control cDNAs derived from a plant not exposed to osmotic stress were subjected to hybridization with individual single cDNAs from a cDNA library of a plant exposed to high salt stress. Thus, constitutively expressed cDNAs in the plant were subtracted, constructing a subtraction library.

More specifically, the control cDNAs were labeled with biotin. Then, they were subjected to hybridization with individual single cDNAs obtained from the cDNA library which comprises cDNAs induced by salt stress. Based on a binding property of biotin to streptavidin, the hybridized cDNAs were removed by applying a membrane coated with streptavidin. Through the course of removal, the residual single cDNAs are genes specifically induced by stress from a high concentration of salt, and were subsequently subjected to sequencing analysis. As a result, some clones of genes expressed by osmotic stress were found. Among the clones found, a new clone OS195 was employed as a probe for screening the Arabidopsis cDNA library. With respect to a nucleotide sequence of the clone, full-length cDNA was isolated. The cDNA has a size of 2090 bp with a putative molecular weight of approximately 61 kDa and an open reading frame encoding 557 amino acids. This was named AtSIK (Arabidopsis thaliana Stress-Inducible Kinase).

A nucleotide sequence of the AtSIK gene is represented by SEQ ID NO: 1, and an amino acid sequence of an AtSIK protein deduced therefrom is represented by SEQ ID NO: 2.

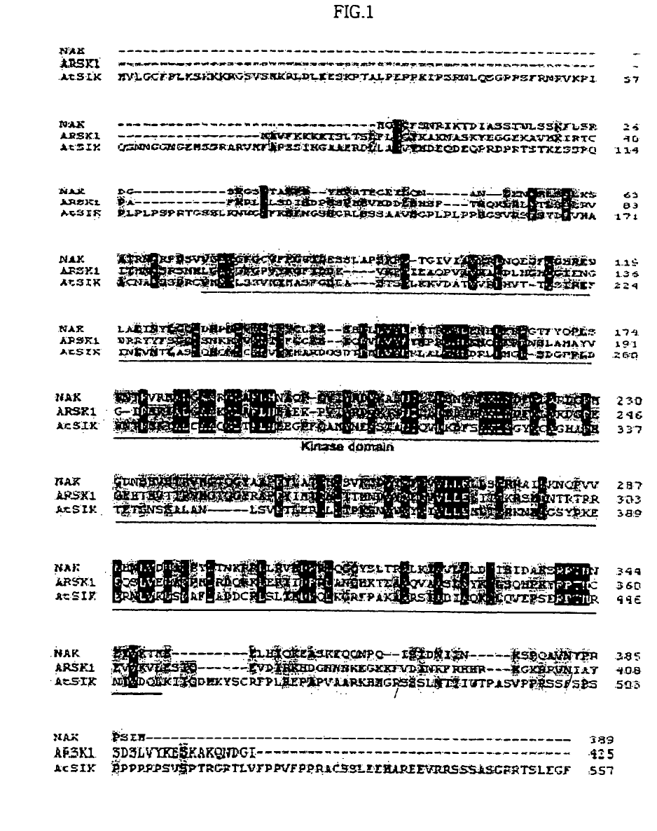

The AtSIK protein has a kinase domain containing 275 amino acids. The kinase domain shows a high amino acid sequence similarity to other kinases such as ARSK1 (Hwang and Goodman, Plant J., 8: 37-43, 1995) and NAK (Moran and Walker, Biochem. Biophys. Acta., 1216: 9-14, 1993). On the other hand, 183 amino acids at the C-terminal end and 104 amino acids at the N-terminal end showed no similarity to those kinases (see FIG. 1). This demonstrates that the C-terminal and the N-terminal of the AtSIK gene are involved in performing AtSIK-specific functions.

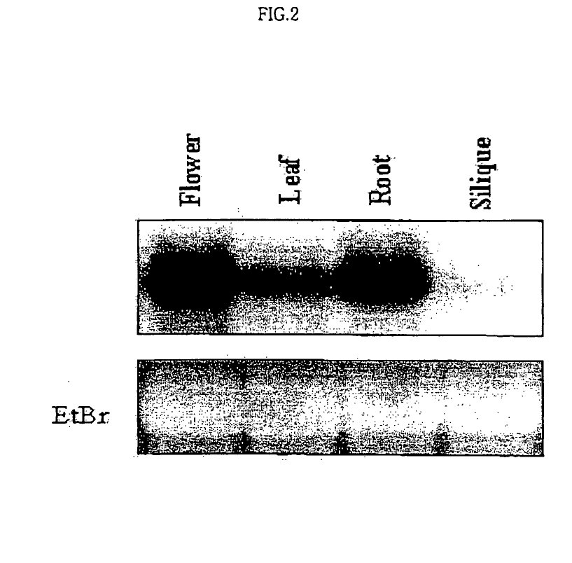

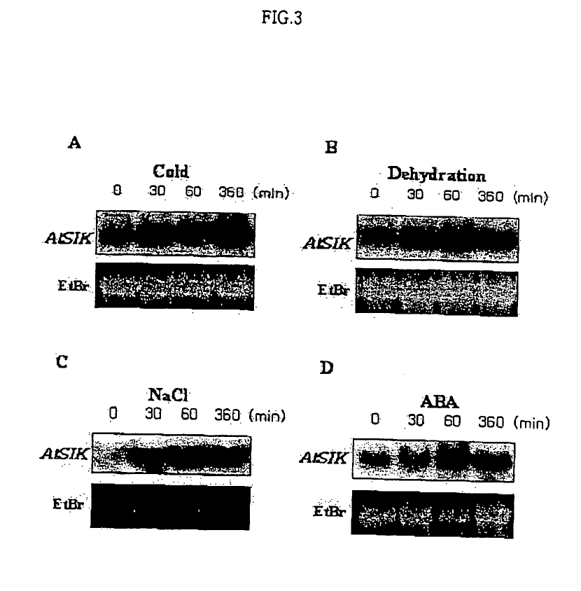

In an embodiment of the invention, with the aim of examining biological functions of the AtSIK protein, total RNAs were respectively isolated from tissues of flowers, leaves, roots, and siliques of Arabidopsis thaliana and subjected to Northern blot analysis. It was seen that AtSIK was expressed at different levels according to the tissues. The flower tissues show the highest level of expression, followed by the root and leaf tissues in order, but there is little expression in silique tissues (see FIG. 2). These results demonstrate that AtSIK expression is regulated at a variable level according to the tissues. Meanwhile, since AtSIK was a gene first isolated from a clone subjected to an osmotic stress, it is necessary to examine its expression patterns according to diverse osmotic stresses. Arabidopsis were exposed to exogenous abscisic acid (ABA) treatment, low temperature, dehydration, and high concentration of salt, respectively, and total RNAs were subjected to Northern blot analysis. The results showed that the expression of AtSIK is induced by all the above treatments. Especially, upon treatment with a high concentration of salt, the gene expression is rapidly induced within 30 min. As for exogenous ABA treatment or dehydration, the expression showed a peak at 60 min after treatment. As for low temperature treatment, the expression peaked at 6 hours after treatment. These results demonstrate that AtSIK is involved in osmotic stress signaling pathway and the expression of AtSIK is regulated at a variable level according to stress conditions (see FIG. 3).

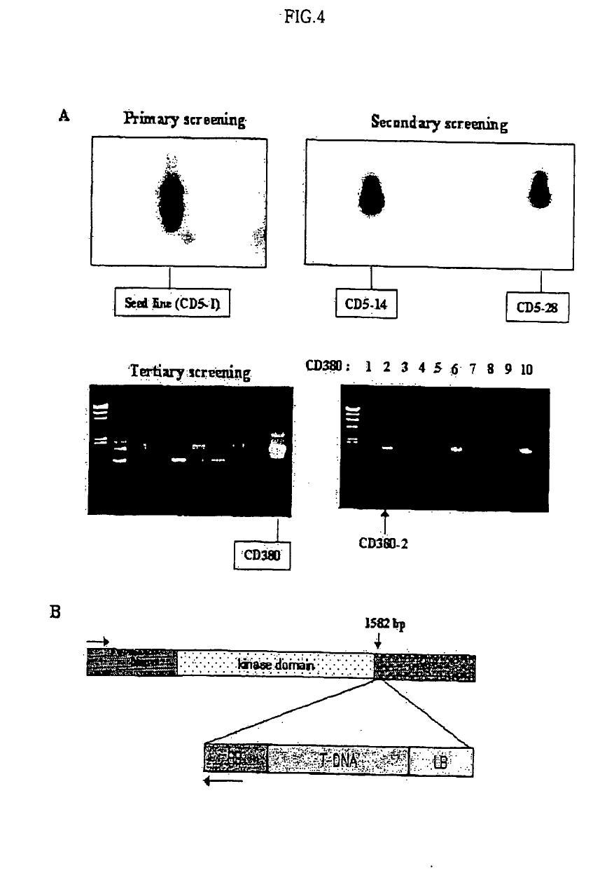

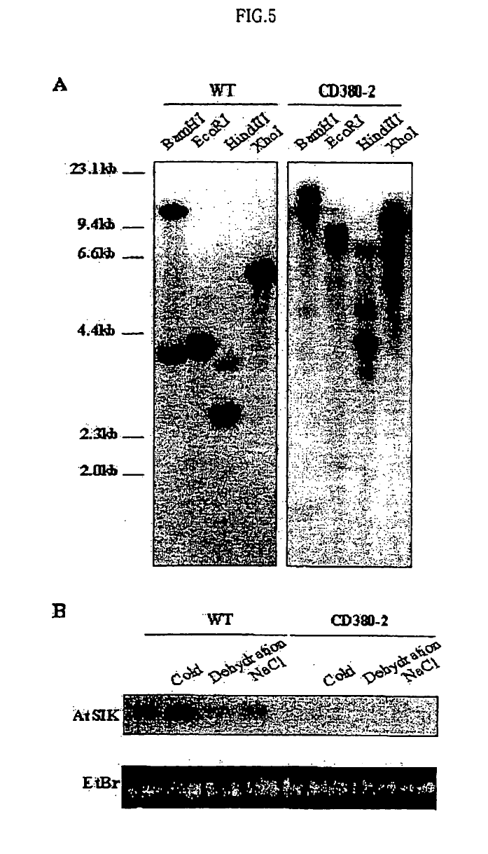

In another embodiment of the invention, with an aim of examining biological roles of AtSIK in response to osmotic stress, the AtSIK gene was inactivated by inserting Agrobacterium T-DNA into AtSIK of Arabidopsis. General methods for inactivating a gene include gene deletion, gene insertion, introduction of antisense strand, T-DNA insertion, homologous recombination, and transposon tagging, and these techniques may be employed for the purpose of the invention. In the invention, an AtSIK gene-inactivated mutant, CD380-2, was isolated by the PCR screening method after T-DNA insertion. In detail, a mutant plant, which harbors a T-DNA insert within AtSIK, was selected from the pooled transgenic plants harboring a T-DNA insert. Using individual genomic DNAs (ABRC, USA) obtained from the group of the transformed plants as templates, PCR was performed using a combination of primers specific for the 3′-end and 5′-end of the AtSIK gene and primers specific for the T-DNA right border (RB) and T-DNA left border (LB). Then, a Southern blot was performed using a ³²P-labeled AtSIK cDNA as a probe for hybridization with the PCR products (see FIG. 4; A). Sequencing was performed on the PCR product to determine whether or not T-DNA is inserted in the genome of the mutant. It was found that T-DNA was inserted at nucleotide 1,582 of the AtSIK cDNA sequence (See FIG. 4; B). DNA was isolated from wild type and the mutant plants, digested with restriction enzymes, followed by Southern blot analysis. The results showed that T-DNA was inserted into AtSIK of the mutant (see FIG. 5; A). In addition, a wild type plant and an AtSIK gene-inactivated plant were exposed to low temperature, dehydration and high salinity, respectively, and expression patterns of AtSIK were determined by Northern blot. The results showed that AtSIK expression was induced only in the wild type plant, not in the mutant plant (see FIG. 5; B). Thus, it was further proven that the mutant plant harbored a T-DNA insert in the AtSIK gene.

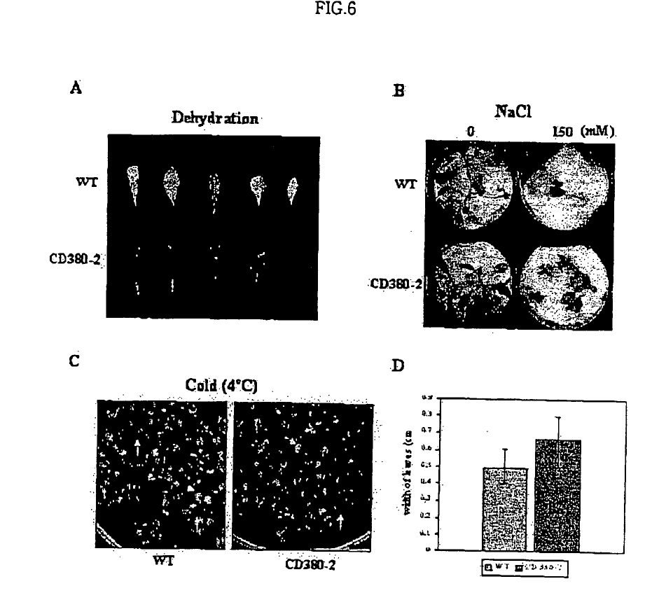

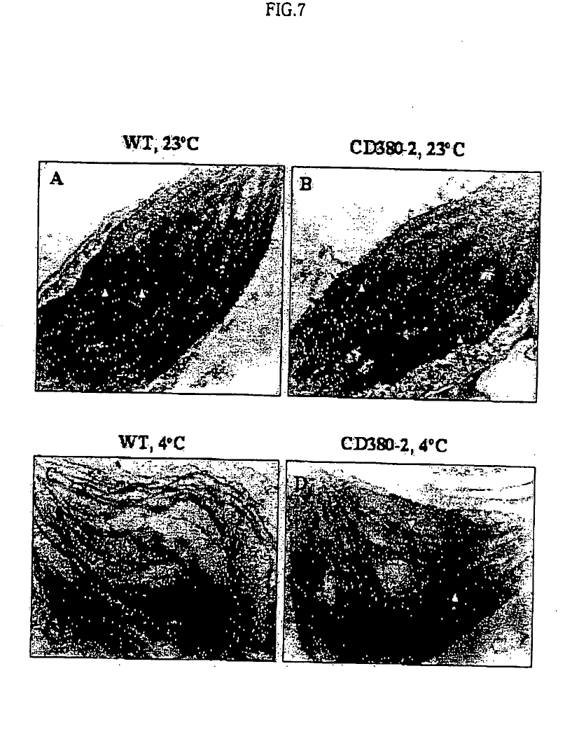

Using the selected mutant plant with the T-DNA insert in AtSIK, physiological roles of the AtSIK gene were examined. Such mutant plants were exposed to a variety of stress conditions, and their phenotypes were examined. The results showed that upon treatment of dehydration or high concentration of salt, leaves of the mutants were less damaged and etiolated, compared to wild type (WT) plants, implying that the mutant has resistance to osmotic stress (see FIG. 6; A and B). The mutant plant also showed resistance to stress by low-temperature treatment (see FIG. 6; C). Where applied with osmotic stress, the mutant leaves were 50% bigger than the wild type leaves (see FIG. 6; D). These results showed that the AtSIK-inactivated mutants have more resistance to osmotic stress than the wild type plants, indicating that AtSIK is involved in exhibiting sensitivity to osmotic stress in plants. Meanwhile, where wild type and mutant plants were exposed to a low temperature of 4° C., and their leaves were finely cut and examined under an electron microscope, a great loss in thylakoid membranes was observed in the wild type plant (see FIG. 7), demonstrating that the AtSIK-inactivated mutants have more resistance to osmotic stress than the wild type plants.

As described above, it was seen that AtSIK-inactivated mutants have resistance to osmotic stress. To study this phenomenon at molecular levels, wild type and mutant plants were applied with diverse osmotic stresses, and expression patterns of osmotic stress-inducible genes in those plants were analyzed. As stress-inducible genes. COR15a, COR47, AtSIZ, and RD29A were of interest. Their expression can be induced by exogenous ABA treatment or a variety of osmotic stresses.

COR15a is one of COR (cold-regulated) genes expressed in plants which undergo cold adaptation, and it is possible that their gene products may be involved in freezing resistance (Artus et al., Proc. Natl. Acad. Sci. USA, 93(23): 13404-13409, 1996). According to a recent report, constitutive expression of COR15a raises a survival rate of Arabidopsis under cold conditions. Cold induces disruption of both the physical continuity and permeability of the plasma membrane, which is responsible for osmotic control. Such damage allows substances of the cytoplasm and organelles to leak out, and causes fusion of the cell membrane with an endomembranes, for example, outer membranes of chloroplasts, thereby lowering freezing resistance. COR15a is a stress-responsive gene raising a survival rate of plants by attenuating responses of the cell membrane and chloroplast membrane to freezing.

Similar to COR15a, COR47 is known as a gene induced by low temperature, ABA and dehydration stresses.

AtSIZ is a polypeptide having C₃H-type zinc finger motif, isolated from Arabidopsis thaliana, and acts as a transcription factor for activating transcription of genes involved in responses of plants to osmotic stress, which is disclosed in Korean Pat. Appln. No. 2000-0072720.

RD29 is a gene of the Arabidopsis plant whose expression is induced by dehydration, cold, or a high concentration of salt. There are two kinds of RD29A and RD29B. A promoter of RD29A exists in most organelles and tissues of plants growing where moisture is deficient, such that the plants have increased resistance to dehydration stress (Yamaguchi et al., Mol. Gen. Genet., 236: 331-340, 1993).

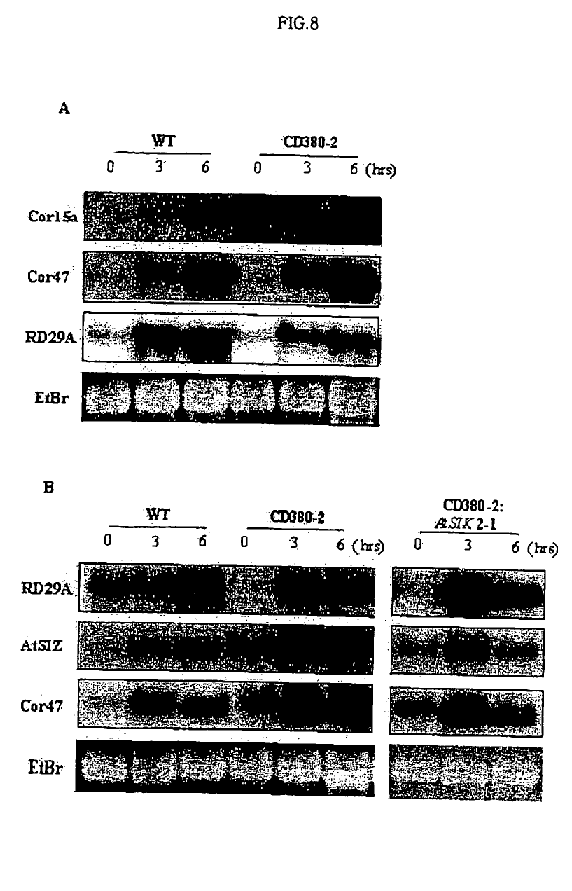

In particular, wild type and mutant plants were exposed to a low temperature, and total RNAs were isolated. Expression patterns of osmotic stress-inducible genes such as COR15a, COR47, AtSIZ, and RD29A were examined by Northern blot analysis. The results showed that although expressions of the genes mentioned above were increased in both wild type and mutant plants, COR15a and COR47 were expressed in large quantities in the mutant plants, compared to the wild type plants (see FIG. 8; A). Where wild type and mutant plants were treated with a high concentration of NaCl, and gene expressions were analyzed by Northern blot, it was also seen that COR47 and AtSIZ were expressed at large quantities in the mutant plants (see FIG. 8; B). However, expression levels of COR15a, COR47 and AtSIZ genes were relatively low in wild type plants. Thus, it was inferred that AtSIK functions to repress genes induced by osmotic stress. However, as for RD29A, expression was induced higher level in the wild type, upon being exposed to a low temperature or a high concentration of salt, compared with the mutant plants, inferring that AtSIK does not repress RD29A gene expression, unlike other osmotic stress-inducible genes. This indicates that AtSIK is not only involved in a negative regulation pathway by which the protein represses osmotic stress-inducible genes in plants, but that it is also involved in a positive regulation pathway.

In yet another embodiment of the invention, a complementation test was carried out to determine whether phenotypic changes of mutant plants are caused by modification of the AtSIK gene due to T-DNA insertion. That is, complementation ability of the mutants was tested to prove that phenotypic changes of the mutant plants, in response to osmotic stress, were attributable to T-DNA insertion in the AtSIK gene.

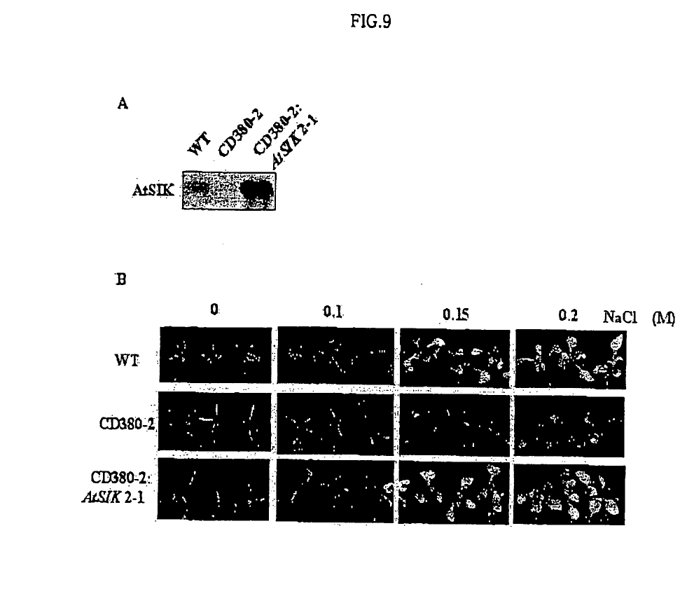

In detail, an AtSIK cDNA was linked to a 35S CaMV promoter of pBIB-HYG, a binary vector carrying a hygromycin resistance marker as a selection marker, so as to construct an AtSIK complement. The complement was introduced to the mutant plant by Agrobacterium-mediated transformation. Transformed plants exhibiting resistance to hygromycin were selected. Then, total RNA was isolated from AtSIK-complemented mutants, AtSIK-inactivated mutants and wild type plants, and AtSIK expression patterns were analyzed by Northern blot. As shown in FIG. 9; A, AtSIK-introduced mutants showed a high level of AtSIK expression. On the other hand, upon treatment with high concentration of salt, phenotypes of wild type, AtSIK-inactivated mutant and AtSIK-complemented mutant plants were examined. AtSIK-complemented mutant plants exhibited sensitivity to high concentration of salt at a level similar to the wild type plants (see FIG. 9; B). This demonstrates that NaCl-resistance phenotype of the AtSIK-inactivated mutants is attributable to a mutation in the AtSIK gene due to T-DNA insertion.

Hereinafter, the present invention will be described in detail, in conjunction with various examples. These examples are provided only for illustrative purposes, and the present invention is not to be construed as being limited to those examples.

### EXAMPLE 1

### Cultivation of Arabidopsis thaliana

All Arabidopsis plants used in experiments of the invention were cultivated in a culture room at 22° C. on Murashige-Skoog (MS) agar plates or a green house controlled to maintain a cycle of 16 hrs light/8 hrs darkness and 70% relative humidity. For a chemical treatment, in 250 ml culture flasks containing MS liquid media under the conditions of 22° C. and a cycle of 16 hrs light/8 hrs darkness while stirring at 1000 rpm. The plant tissues of choice were collected and immediately frozen under liquid nitrogen.

### EXAMPLE 2

### Isolation of Osmotic Stress-Inducible Genes

To isolate genes whose expressions are induced by osmotic stress, especially, salt stress, a subtraction library was constructed, and then cDNAs were randomly selected from the library and screened by nucleotide sequencing. For the subtraction library, cDNAs derived from plants exposed to salt stress were subjected to hybridization with cDNAs derived from the control plant which was not exposed to salt stress, thereby subtracting the constitutively expressed cDNAs.

2-1: Culture of the Arabidopsis Plant

The Arabidopsis plants were cultured under the same conditions as in Example 1. After 1 week, with the purpose of obtaining stress-inducible cDNAs, the seedlings were exposed to salt stress by exchanging the media with MS media containing 0.15 M NaCl. The seedlings were cultured for an additional 1 to 6 hrs while stirring. All seedlings were immediately frozen and stored at −80° C.

2-2: Extraction of RNA and Construction of cDNA Library

To prepare an osmotic stress-inducible cDNA library, total RNA was first extracted from a frozen seedling which had been treated with 0.15 M NaCl for 6 hrs, using a lithium chloride/phenol extraction method. The total RNA was treated according to the protocol of an mRNA isolation kit (Pharmacia, USA) to isolate poly(A)⁺ RNA. As a control, salt-untreated seedlings were subjected to the above procedure to isolate poly(A)⁺ RNA. Then, osmotic stress-inducible cDNAs were prepared using a cDNA synthesis kit and poly(A)⁺ RNA (Stratagene, USA). The individual cDNAs were ligated to λ ZAP II (Stratagene, USA), finally constructing a λ ZAP II cDNA library.

2-3: Construction of Subtraction Library

With regard to the λ ZAP II cDNA library, a quantity of filamentous phages was obtained, and individual single stranded DNAs were purified therefrom. To remove constitutively expressed cDNAs from the λ ZAP II cDNA library derived from the stress-applied plant, subtraction was performed by hybridization with an excess amount of the control cDNA.

To label the control cDNA with biotin, an excess amount of double stranded cDNA prepared above was subjected to PCR incorporating biotinylated dUTP.

In detail, 100 ng of the control double stranded cDNA was employed as a template. As for primers, oligonucleotides having the nucleotide sequences of SEQ ID NO: 3 and SEQ ID NO: 4, respectively, were synthesized and 50 ng of each were employed. 50 μM of biotin-16-dUTP was supplied in a PCR reaction mixture, thereby producing biotin-labeled cDNA. The PCR condition was 30 sec at 94° C., 30 sec at 38° C., 30 sec at 72° C., and 50 cycles thereof.

To subtract constitutively expressed cDNAs, hybridization was carried out. 0.1 μg of the single stranded cDNA from the λ ZAP II cDNA library and 1 μg of the biotinylated control cDNA probe were added to a 100 μl of hybridization cocktail (containing 50 mM Tris-HCl, pH 7.5, 0.25 M NaCl and 1.0 mM EDTA) at 65° C. overnight. The hybridization solution was incubated with membranes coated with streptavidin on ice for 2 hrs with occasional stirring, followed by removing the membranes. Since streptavidin has a high affinity for biotin, cDNA samples hybridized with the biotinylated control cDNA can be removed by eliminating the membranes coated with streptavidin. That is, after hybridization, only cDNAs which are specifically expressed by salt stress remained in the hybridization solution. These cDNAs were extracted by employing phenol/chloroform and chloroform. Then, the cDNAs were added with 2 μg carrier tRNA and cold ethanol at −20° C., thereby being precipitated. The cDNAs thus obtained were introduced to an E. coli host cell by an electroporation method. In this way, the subtraction library was prepared. From such a library, clones were randomly selected, and their DNA sequences were analyzed using an automatic sequencer.

From the sequencing analysis, 15 osmotic stress-inducible ESTs (Expressed Sequence Tags) were found. Among these, 4 cDNAs were found to be novel genes. Specifically, the clone carrying a 0.8 kb DNA was employed in experiments thereafter.

### EXAMPLE 3

### Isolation of AtSIK cDNA

The cDNA clones whose gene expression is regulated according to varying osmotic environment were isolated as described above, for understanding mechanisms of the responses of plants to osmotic stress. Full-length cDNA was isolated from the Arabidopsis cDNA library by employing a new clone OS195 as a probe. The cDNA has a size of 2090 bp with a putative molecular weight of approximately 61 kDa and an open reading frame encoding 557 amino acids. This was named AtSIK (Arabidopsis thaliana Stress-Inducible Kinase). The full-length cDNA was subcloned to pBluescript vector, constructing a recombinant plasmid. E. coli transformed with the said recombinant plasmid was deposited in the Korean Collection for Type Cultures (KCTC) affiliated with the Korea Research Institute of Bioscience and Biotechnology (KRIBB), under deposit No. KCTC 0932BP on Jan. 9, 2001.

The AtSIK protein has a kinase domain containing 275 amino acids. An amino acid sequence of AtSIK protein, which is deduced from the full-length cDNA, was subjected to sequence comparison using a BlastX program of NCBI. The results showed that the kinase domain has a high amino acid sequence similarity to other kinases such as ARSK1 (Hwang and Goodman, Plant J., 8: 37-43, 1995) and NAK (Moran and Walker, Biochem. Biophys. Acta., 1216: 9-14, 1993). On the other hand, 183 amino acids at the C-terminal end and 104 amino acids at the N-terminal end showed no similarity to those kinases (see FIG. 1). This demonstrates that the C-terminal and the N-terminal of the AtSIK gene are involved in performing AtSIK-specific functions.

### EXAMPLE 4

### Induction of AtSIK Expression by Diverse Osmotic Stresses

To determine biological functions of AtSIK, expression patterns of AtSIK by diverse osmotic stresses in diverse tissues were examined.

4-1: Expression Patterns in Diverse Tissues

Total RNAs were isolated from tissues of flowers, leaves, roots and siliques of Arabidopsis thaliana, respectively, using a LiCl/phenol method. Northern blotting was performed to assess the transcription levels in these tissues, employing AtSIK cDNA as a probe. 15 μg total RNA from each tissue was heat-treated at 65° C. for 15 min to loosen its secondary structure, and mixed with formaldehyde gel loading buffer (50% glycerol, 1 mM EDTA, pH 8.0, 0.25% bromophenol blue, 0.25% xylene cyanol FF in distilled water). Each sample was loaded on a 1% agarose gel containing 2.2 M formaldehyde, followed by slow electrophoresis at a voltage of 4 V/cm. The gel with developed RNAs was immersed in DEPC-H₂O to remove formaldehyde. Then, the gel was transferred to a nylon membrane by capillary transfer for about 16 hrs, followed by heat treatment at 80° C. for 1 hr, thereby immobilizing RNAs. As a probe for hybridization, AtSIK cDNA was labeled with [α-³²P] dCTP with the aid of a random primer labeling kit (Boehringer Mannheim, Germany). That the same amount of respective total RNAs was loaded into each well in the gel was confirmed by staining with ethidium bromide (EtBr). The RNAs-loaded gel was immersed in a solution containing EtBr for 30 min while stirring. Finally, for the detection of hybridization, the membrane was exposed on X-ray film at −70° C. The results are shown in FIG. 2. It was seen that AtSIK is expressed at different levels according to the tissues. The flower tissues show the highest level of expression, followed by the root and leaf tissues in order, but there is little expression in silique tissues. These results demonstrate that AtSIK expression is regulated at a variable level according to tissue type.

4-2: Expression Patterns According to Osmotic Stress Variety

Expression of AtSIK under a variety of osmotic stress was examined. Arabidopsis plants were exposed to high concentration of salt (NaCl), dehydration, low temperature, and exogenous abscisic acid (ABA), respectively. Total RNAs were isolated and subjected to Northern blot analysis using a method analogous to Example 4-1. The results are shown in FIG. 3. The expression of AtSIK was induced by all the above treatments, indicating that AtSIK is involved in responses to general osmotic stress. Despite such a common expression of AtSIK, its expression pattern was different according to the stress conditions. As for the low temperature treatment, the expression was induced at a relatively high level after 30 min, with a peak at 6 hours after treatment. On the other hand, as for treatment of dehydration, high concentration of salt and exogenous ABA treatment, the expression showed a peak at 60 min after treatment, followed by a decrease after 6 hrs. These results demonstrate that the expression of AtSIK is regulated at a variable level according to stress conditions.

### EXAMPLE 5

### Isolation of an Arabidopsis Mutant Plant with T-DNA Inserted in AtSIK Gene

It is necessary to compare a wild type plant with an AtSIK gene-inactivated plant for understanding biological roles of AtSIK.

To select a mutant having T-DNA inserted within an AtSIK gene, a PCR screening method (McKinney et al., Plant J., 4: 613-622, 1995) was used to probe genomic DNAs (ABRC, USA) of a group of transformed plants harboring a T-DNA tag. Three rounds of PCR screening were performed using a combination of primers specific for the 3′-end and 5′-end (SEQ ID NO: 5 and SEQ ID NO: 6, respectively) of the AtSIK gene and primers specific for the T-DNA right border (RB) and T-DNA left border (LB) (SEQ ID NO: 7 and SEQ ID NO: 8, respectively). As for respective templates, individual genomic DNAs (ABRC, USA) obtained from a group of transformed plants harboring T-DNA inserts were employed. The PCR product thus obtained was subjected to Southern blot analysis employing a 32P labeled AtSIK cDNA as a probe for detecting hybridization (see FIG. 4; A). To prove that said PCR product is identical to the AtSIK gene, and to determine the location of the T-DNA insertion, the PCR product was subcloned to a pBluescript vector, followed by sequencing. It was found that T-DNA was inserted at nucleotide 1582 of the AtSIK cDNA sequence (See FIG. 4; B).

Disruption of the AtSIK gene in such a transformed plant was confirmed by digesting the genomic DNA obtained therefrom with BamHI, EcoRI, HindIII, and XhoI, followed by Southern blot analysis (see FIG. 5; A). In addition, a wild type plant and an AtSIK gene-inactivated plant were exposed to low temperature, dehydration and NaCl, respectively, and expression patterns of AtSIK were determined by Northern blot. The results showed that no AtSIK expression was induced in transformed plants (see FIG. 5; B). Thus, disruption of the AtSIK gene in the transformed plant was further proven.

### EXAMPLE 6

### Determination of Phenotype of T-DNA Insertion Mutant

Phenotypes of mutant plants in diverse environments were examined. For low-temperature treatment, Arabidopsis wild type and mutant plants were grown on an MS plate at 23° C. for 1 week. The plate was transferred to a culture room at 4° C. and cultured for 3 months. Effects of high concentrations of salt were examined. 10 day-old plants were transferred to MS plates containing varying concentrations of salts, and the phenotype was observed for 4 days. For treatment of dehydration, 4 week-old plants were transferred to plates without water, followed by incubation for 10 days. The results showed that upon treatment of dehydration or high concentration of salt, leaves of the mutants were less damaged and etiolated compared to the wild type (WT) plants, implying that the mutants have improved resistance to osmotic stress (see FIG. 6; A and B). The mutant plants also showed resistance to stress due to low-temperature treatment (see FIG. 6; C). Where applied with osmotic stress, the mutants had leaves 50% bigger than the wild type plants (see FIG. 6; D). In addition, as for low-temperature treated WT and mutant plants, ultrathin sections were prepared from the seed leaves and rosette leaves, and their thylakoid membranes were examined under an electron microscope. No changes in structures of the thylakoid membranes in the mutant plants were seen, compared to the untreated plants, while such thylakoid membranes were not seen in the wild type plants (see FIG. 7). From the above results, it can be seen that the AtSIK-inactivated mutants have more resistance to osmotic stress than the wild type plants.

### EXAMPLE 7

### Regulatory Effect of AtSIK on Repression of Stress-Responsive Genes

On the basis of the experimental results showing that AtSIK-inactivated mutants have resistance to osmotic stress, molecular biological studies thereon were conducted. The wild type and mutant plants were applied with osmotic stress. Expression patterns of osmotic stress-inducible genes such as COR15a, COR47, RD29A, and AtSIZ were examined by Northern blot analyses. First, wild type, mutant and AtSIK-complemented mutant plants were cultured in liquid MS media for 1 week, and then the plants were exposed to 150 mM NaCl or a low temperature of 4° C., each for both 3 hrs and 6 hrs. As stress-inducible genes, COR15a, COR47, RD29A, and AtSIZ served as reporter genes. Their expression can be regulated by exogenous ABA treatment or a variety of osmotic stresses. Northern blot analysis was performed employing ³²P-labeled cDNAs of COR15a, COR47, RD29A, and AtSIZ, as probes. The results showed that COR15a and COR47 are expressed in large quantities in the mutant plants exposed to low temperature (see FIG. 8; A). It was also seen that COR47 and AtSIZ are expressed in large quantities in the mutant plants exposed to 150 mM NaCl (see FIG. 8; B). Thus, it was inferred that AtSIK functions to repress genes induced by osmotic stress. However, as for RD29A, the wild type plants showed higher expression upon being exposed to low temperature or high concentration of salt than the mutant plants, implying that AtSIK induces RD29A gene expression.

### EXAMPLE 8

### Complementation Test

The mutant plants were isolated from transgenic lines inserted with T-DNA. Therefore, complementation ability of the mutants was tested to prove that such osmotic stress-resistant phenotypes of the mutant plants were attributable to insertion of T-DNA into the AtSIK gene.

8-1: Construction of an AtSIK Complement and Production of an AtSIK-Complemented Mutant Using the Complement

An AtSIK cDNA was inserted in pBIB-HYG, a binary vector carrying a hygromycin resistance marker gene as a selection marker (Becker D., Institut fir Genetic der Universitat zu Koln, FRG, Nucleic Acid Res., 180(1): 203, 1990) so as to construct an AtSIK complement. First, the AtSIK subcloned in pBluescript was digested with EcoRI and XhoI, followed by filling in the 5-protruding ends using a mixture of Klenow fragment and dNTPs to make a blunt ended DNA. Thus, an AtSIK insert was obtained. The insert was ligated into an Ecl136II restriction enzyme site of pBIB-HYG vector, ensuring that the insert is located between the CaMV 35S promoter and the NOS terminator, thereby constructing a recombinant vector capable of expressing an AtSIK protein. The recombinant vector thus constructed was introduced to a mutant plant harboring an inactivated AtSIK gene by an Agrobacterium-mediated vacuum infiltration method, producing transformed plants. The transformed plants were selected on a MS plate containing 50 mg/ml hygromycin. Such selected plants were transferred to soil, and their seeds were obtained. These seeds were again selected on a hygromycin-containing MS plate. In this way, homozygous T2 lines of AtSIK-complemented mutants were obtained. Total RNA was extracted from wild type, mutant and the AtSIK-complemented homozygous T2 mutant plants, followed by Northern blot analysis employing ³²P-labeled AtSIK cDNA as a probe. FIG. 9; A, shows that the AtSIK-complemented homozygous T2 mutant line 2-1 contains large amounts of transcripts of the AtSIK gene, while the AtSIK-inactivated mutant contains few of the transcripts.

8-2: NaCl Stress Sensitivity Test of AtSIK-Complemented Mutant Plants

The sensitivity of AtSIK-complemented homozygous mutant plants to NaCl stress at various NaCl concentrations were tested to determine whether homozygous T2 AtSIK-inactivated mutant plants were complemented by the AtSIK complement. First, wild type, mutant and AtSIK-complemented homozygous T2 mutant (line 2-1) plants were cultivated in soil for 3 weeks. The plants were transferred to plates and cultivated for 10 days. Then, the plants were again transferred to plates containing 0, 0.1, 0.15, 0.2 M NaCl solutions, respectively, and cultivated for 4 days. As shown in FIG. 9; B, the AtSIK-complemented homozygous mutant plants exhibited sensitivity to high concentrations of salts at a level similar to the wild type plants. This demonstrates that NaCl-resistance phenotype of the AtSIK-inactivated mutants is attributable to a mutation in the AtSIK gene due to T-DNA insertion.

### INDUSTRIAL APPLICABILITY

As apparent from the above description, the present inventors discovered a novel protein AtSIK having a function as a negative regulator of genes induced by osmotic stress, and a gene encoding the AtSIK, and found that the AtSIK gene represses genes involved in resistance to osmotic stress in plants. In other words, mutant plants having a mutation in the gene exhibit resistance to osmotic stress, while wild type plants having no mutation in the gene are susceptible to osmotic stress. Accordingly, it is possible to transform a plant with a recombinant vector carrying the AtSIK gene, and then introduce an Agrobacterium T-DNA thereto, thereby constructing a plant in which functions of AtSIK are inactivated. Consequently, such a plant can induce overexpression of genes involved in exhibiting resistance to osmotic stress, thereby accomplishing a considerable increase in plant productivity.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A protein comprising the amino acid sequence of SEQ ID NO: 2.

2. The protein according to claim 1, wherein the protein is isolated from Arabidopsis thaliana.

3. The protein according to claim 1, wherein the protein has a kinase domain, and functional domains for repressing osmotic stress-inducible genes at the C-terminal region and the N-terminal region.

4. A gene encoding the protein according to claim 1.

5. The gene according to claim 4, wherein the gene comprises the AtSIK gene having the nucleotide sequence of SEQ ID NO: 1.

6. An eukaryotic expression vector comprising the gene according to claim 5.

7. The eukaryotic expression vector according to claim 6, wherein the vector comprises the AtSIK gene having the nucleotide sequence of SEQ ID NO: 1 (deposit number: KCTC 0932BP).

8. A method for enhancing resistance to osmotic stress in a plant, comprising the step of inactivating the gene according to claim 5, thereby repressing production of the protein according to claim 1.

9. The method according to claim 8, wherein the step of inactivating the gene is selected from the group consisting of gene deletion, gene insertions introduction of antisense strand, T-DNA insertion, homologous recombination, and transposon tagging.

10. The method according to claim 8, wherein the osmotic stress is the stress caused by high concentration of salt, low temperature, dehydration or abscisic acid treatment.
